(19) United States
(12) Patent Application Publication
(10) Pub. No.: US 2019/0350555 A1
(43) Pub. Date: Nov. 21, 2019

(54) ULTRASONIC TRANSDUCER, ULTRASONIC ENDOSCOPE, AND METHOD OF MANUFACTURING ULTRASONIC TRANSDUCER

(71) Applicant: OLYMPUS CORPORATION, Tokyo (JP)

(72) Inventor: Katsuhiro WAKABAYASHI, Tokyo (JP)

(73) Assignee: OLYMPUS CORPORATION, Tokyo (JP)

(21) Appl. No.: 16/529,878

(22) Filed: Aug. 2, 2019

Related U.S. Application Data
(63) Continuation of application No. PCT/JP2017/039194, filed on Oct. 30, 2017.

(51) Int. Cl. A61B 8/00; B06B 1/02; B06B 1/06; H01L 41/04; H01L 41/047; H01L 41/08; H01L 41/333; H01L 41/338; A61B 8/12

(52) U.S. Cl. CPC A61B 8/4488; B06B 1/0215; B06B 1/0633; B06B 1/067; H01L 41/042; H01L 41/0475; H01L 41/1875; H01L 41/333; H01L 41/338; A61B 8/4494; A61B 8/12; B06B 2201/76; H01L 41/0825

(57) ABSTRACT

An ultrasonic transducer includes a piezoelectric element that extends in a predetermined direction; a first electrode formed on a first surface of the piezoelectric element, in parallel with the direction, the first electrode including: a first portion for inputting an electrical signal to the piezoelectric element, and a first connection portion formed continuously with the first portion and intersecting the direction, wherein a first wiring is electrically connected to the first connection portion; and a second electrode disposed on a second surface, oppose to the first surface, of the piezoelectric element and spaced apart from the first electrode in the piezoelectric element, the second electrode including: a second portion for inputting an electric signal to the piezoelectric element, and a second connection portion formed continuously with the second portion, wherein a second wiring is electrically connected to the second connection portion and collectively arranged with the first wiring.

### CROSS-REFERENCE TO RELATED APPLICATION

This application is a continuation of International Application No. PCT/JP2017/039194, filed on Oct. 30, 2017, the entire contents of which are incorporated herein by reference.

### BACKGROUND

The present disclosure relates to an ultrasonic transducer, an ultrasonic endoscope, and a method of manufacturing the ultrasonic transducer.

An ultrasonic endoscope that inserts a flexible elongated insertion portion into a subject, such as a human being, and observes the inside of the subject by using an ultrasonic transducer disposed at a distal end of the insertion portion has been known in the past (for example, see Japanese Laid-open Patent Publication No. 2009-297118 A, referred to as JP 2009-297118 A).

The ultrasonic transducer described in JP 2009-297118 A is constructed by an ultrasonic transducer of an electronic radial scan system, and includes a plurality of piezoelectric elements that are regularly arrayed so as to form a cylinder. More specifically, the ultrasonic transducer is configured such that a backing member, the piezoelectric elements, and an acoustic matching layer are laminated in an integrated manner and an acoustic lens is mounted on an outer surface side of the acoustic matching layer.

Here, each of the piezoelectric elements includes a piezoelectric body that has a flat plate shape, an upper electrode that is disposed on one plate surface of the piezoelectric body on an outer surface side of the ultrasonic transducer, and a lower electrode that is disposed on the other plate surface of the piezoelectric body. Further, a lead wire is electrically connected to each of the upper electrode and the lower electrode. Then, ultrasonic waves that are generated by the piezoelectric elements in accordance with electrical signals input via the lead wires are condensed by the acoustic lens via the acoustic matching layer and applied to the inside of the subject. Furthermore, ultrasonic waves reflected inside the subject enter the piezoelectric elements via the acoustic lens and the acoustic matching layer, converted to electrical signals by the piezoelectric elements, and output via the lead wires.

Meanwhile, when a joint portion between each of the upper electrodes and the lower electrodes and each of the lead wires is located close to the acoustic lens, the joint portions may be corroded by a medical agent or the like that has transmitted through the acoustic lens, and electrical connections between the upper and lower electrodes and the corresponding lead wires may be disconnected.

Therefore, the ultrasonic transducer described in JP 2009-297118 A adopts a configuration as described below in order to improve durability against a medical agent or the like. Meanwhile, a “proximal end side” described below indicates a side away from the distal end of the insertion portion.

The upper electrode and the lower electrode have longer lengths than the piezoelectric body in an insertion axis direction along an extending direction of the insertion portion, and protrude to the proximal end side relative to the piezoelectric body. Further, a dummy substrate that has the same thickness as the piezoelectric body but does not have a piezoelectric function is sandwiched between protruding portions of the upper electrode and the lower electrode on the proximal end side. Furthermore, a wiring protective layer is disposed adjacent to the proximal end side of the acoustic matching layer such that the upper electrode is sandwiched between the wiring protective layer and the dummy substrate. A boundary between the wiring protective layer and the acoustic matching layer is located on the proximal end side relative to a proximal end portion of the acoustic lens. Each of the lead wires is electrically connected to the protruding portion of each of the upper electrode and the lower electrode on the proximal end side, at a position on the proximal end side relative to the boundary between the wiring protective layer and the acoustic matching layer.

In the configuration as described above, a path length of an entry pathway of a medical agent or the like from the acoustic lens to the joint portion between each of the upper electrode and the lower electrode and each of the lead wires via the boundary between the wiring protective layer and the acoustic matching layer is increased, so that the medical agent or the like is less likely to reach the joint portion between the upper and lower electrodes and the corresponding lead wires. In other words, it is possible to improve durability against a medical agent or the like.

### SUMMARY

The present disclosure is directed to an ultrasonic transducer, an ultrasonic endoscope, and a method of manufacturing the ultrasonic transducer.

According to a first aspect of the present disclosure, an ultrasonic transducer that transmits and receives an ultrasonic wave is provided. The ultrasonic transducer includes a piezoelectric element that extends in a first direction and emits an ultrasonic wave in accordance with an input electrical signal, and converts an ultrasonic wave that is input from outside into an electrical signal; a first electrode that is formed on a first surface of the piezoelectric element to be in parallel with the first direction, the first electrode including: a first input portion through which the input electrical signal is to be input to the piezoelectric element, and a first connection portion being formed continuously with the first input portion and intersecting the first direction, wherein a first wiring is electrically connected to the first connection portion; and a second electrode that is formed on a second surface of the piezoelectric element to be spaced apart from the first electrode, the second surface being oppose to the first surface in the piezoelectric element, the second electrode including: a second input portion through which the input electrical signal is to be input to the piezoelectric element, and a second connection portion being formed continuously with the second input portion, wherein a second wiring is electrically connected to the second connection portion and arranged collectively with the first wiring.

According to a second aspect of the present disclosure, an ultrasonic transducer unit is provided which includes the ultrasonic transducer according to the first aspect; and a flexible printed substrate including an electrically insulating base material and an electrically conductive layer that serves as at least one of the first wiring and the second wiring, wherein the electrically conductive layer is bent with respect to the base material and electrically connected to either one of the first electrode and the second electrode.

According to a third aspect of the present disclosure, an ultrasonic endoscope is provided which an insertion portion to be inserted into a subject to be examined; and the ultrasonic transducer unit according to the second aspect, the ultrasonic transducer unit being provided at a distal end portion of the insertion portion.

According to a fourth aspect of the present disclosure, a method of manufacturing an ultrasonic transducer is provided. The method includes electrically connecting a first wiring on a portion of a first thin film that is continuously formed on a first surface and an intersecting surface of a piezoelectric element base material to constitute a piezoelectric element, the intersecting surface intersecting with the first surface, the portion corresponding to a first connection portion provided on the intersecting surface, the first thin film being to serve as a first electrode; electrically connecting a second wiring on a second thin film formed on a second surface oppose to the first surface of the piezoelectric element base material, the second thin film being to serve as a second electrode; manufacturing a molding member by forming a laminating member on one of the first thin film and the second thin film; and producing a plurality of the piezoelectric elements by cutting the molding member to a middle depth of the laminating member after electrically connecting the first wiring, electrically connecting the second wiring, and manufacturing the molding member, wherein at least one of the first wiring and the second wiring is an electrically conductive layer provided on a flexible printed substrate.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an endoscope system according to an embodiment;

FIG. 2 is a diagram illustrating a configuration of an ultrasonic transducer;

FIG. 3 is a diagram illustrating the configuration of the ultrasonic transducer;

FIG. 4 is a diagram illustrating the configuration of the ultrasonic transducer;

FIG. 5 is a diagram illustrating the configuration of the ultrasonic transducer;

FIG. 6 is a flowchart illustrating a method of manufacturing the ultrasonic transducer;

FIG. 7A is a diagram for explaining the method of manufacturing the ultrasonic transducer;

FIG. 7B is a diagram for explaining the method of manufacturing the ultrasonic transducer;

FIG. 7C is a diagram for explaining the method of manufacturing the ultrasonic transducer;

FIG. 7D is a diagram for explaining the method of manufacturing the ultrasonic transducer;

FIG. 7E is a diagram for explaining the method of manufacturing the ultrasonic transducer;

FIG. 7F is a diagram for explaining the method of manufacturing the ultrasonic transducer;

FIG. 7G is a diagram for explaining the method of manufacturing the ultrasonic transducer;

FIG. 8 is a diagram illustrating an ultrasonic transducer according to a first modification of the embodiment;

FIG. 9 is a diagram illustrating an ultrasonic transducer according to a second modification of the embodiment;

FIG. 10 is a diagram illustrating an ultrasonic transducer according to a third modification of the embodiment; and

FIG. 11 is a diagram illustrating an ultrasonic transducer according to a fourth modification of the embodiment.

### DETAILED DESCRIPTION

Modes for carrying out the present disclosure (hereinafter, referred to as embodiments) will be described below with reference to the drawings. The present disclosure is not limited by the embodiments described below. Further, in the description of the drawings, the same components are denoted by the same reference symbols.

Schematic Configuration of Endoscope System

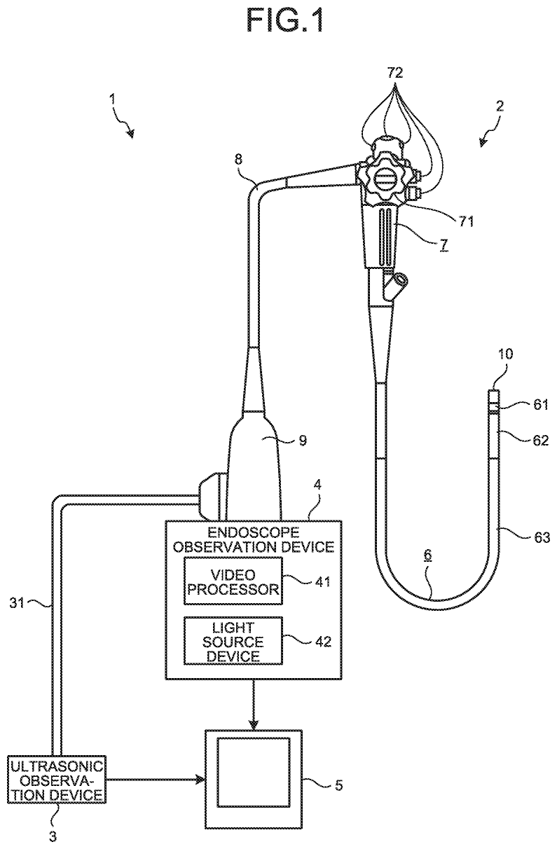

FIG. 1 is a diagram schematically illustrating an endoscope system 1 according to an embodiment.

The endoscope system 1 is a system that performs ultrasonic diagnosis inside a subject, such as a human being, using an ultrasonic endoscope. As illustrated in FIG. 1, the endoscope system 1 includes an ultrasonic endoscope 2, an ultrasonic observation device 3, an endoscope observation device 4, and a display device 5.

The ultrasonic endoscope 2 includes a portion that can be inserted into the subject, has a function to transmit ultrasonic pulses (acoustic pulses) toward a body wall inside the subject, receive ultrasonic echoes reflected by the subject, and output an echo signal, and has a function to capture an image inside the subject and output an image signal.

A detailed configuration of the ultrasonic endoscope 2 will be described later.

The ultrasonic observation device 3 is electrically connected to the ultrasonic endoscope 2 via an ultrasonic cable 31 (FIG. 1), outputs a pulse signal to the ultrasonic endoscope 2 via the ultrasonic cable 31, and receives input of an echo signal from the ultrasonic endoscope 2. Then, the ultrasonic observation device 3 performs predetermined processing on the echo signal and generates an ultrasonic image.

An endoscope connector 9 (FIG. 1) (to be described later) of the ultrasonic endoscope 2 is removably connected to the endoscope observation device 4. As illustrated in FIG. 1, the endoscope observation device 4 includes a video processor 41 and a light source device 42.

The video processor 41 receives an image signal from the ultrasonic endoscope 2 via the endoscope connector 9. Then, the video processor 41 performs predetermined processing on the image signal and generates an endoscopic image.

The light source device 42 supplies illumination light for illuminating the inside of the subject to the ultrasonic endoscope 2 via the endoscope connector 9.

The display device 5 is constructed with liquid crystal or organic electro luminescence (EL), and displays the ultrasonic image generated by the ultrasonic observation device 3, the endoscopic image generated by the endoscope observation device 4, and the like.

Configuration of Ultrasonic Endoscope

Next, a configuration of the ultrasonic endoscope 2 will be described.

As illustrated in FIG. 1, the ultrasonic endoscope 2 includes an insertion portion 6, an operating unit 7, a universal cord 8, and the endoscope connector 9.

Meanwhile, a “distal end side” described below indicates a distal end side of the insertion portion 6 (a distal end side in an insertion direction into the subject). Further, a “proximal end side” described below indicates a side away from the distal end of the insertion portion 6.

The insertion portion 6 is a portion to be inserted into the subject. As illustrated in FIG. 1, the insertion portion 6 includes an ultrasonic transducer 10 that is disposed on the distal end side, a rigid member 61 that is connected to a proximal end side of the ultrasonic transducer 10, a bending portion 62 that is connected to a proximal end side of the rigid member 61 and is bendable, and a flexible tube 63 that is connected to a proximal end side of the bending portion 62 and has flexibility.

A detailed configuration of the ultrasonic transducer 10 that is a main component of the present disclosure will be described later.

The operating unit 7 is a portion that is connected to a proximal end side of the insertion portion 6 and receives various kinds of operation from a doctor or the like. As illustrated in FIG. 1, the operating unit 7 includes a bending knob 71 for performing bending operation on the bending portion 62, and a plurality of operating members 72 for performing various kinds of operation.

The universal cord 8 is a cord which is extended from the operating unit 7 and in which a light guide (not illustrated) for transmitting the illumination light supplied by the light source device 42, a transducer cable (not illustrated) for transmitting the pulse signal and the echo signal as described above, a signal cable (not illustrated) for transmitting the image signal as described above, and the like are arranged.

The endoscope connector 9 is disposed on an end portion of the universal cord 8. The ultrasonic cable 31 is connected to the endoscope connector 9. The ultrasonic cable 31 is inserted in the endoscope observation device 4 and thus connected to the video processor 41 and the light source device 42.

Configuration of Ultrasonic Transducer

Next, a configuration of the ultrasonic transducer 10 will be described.

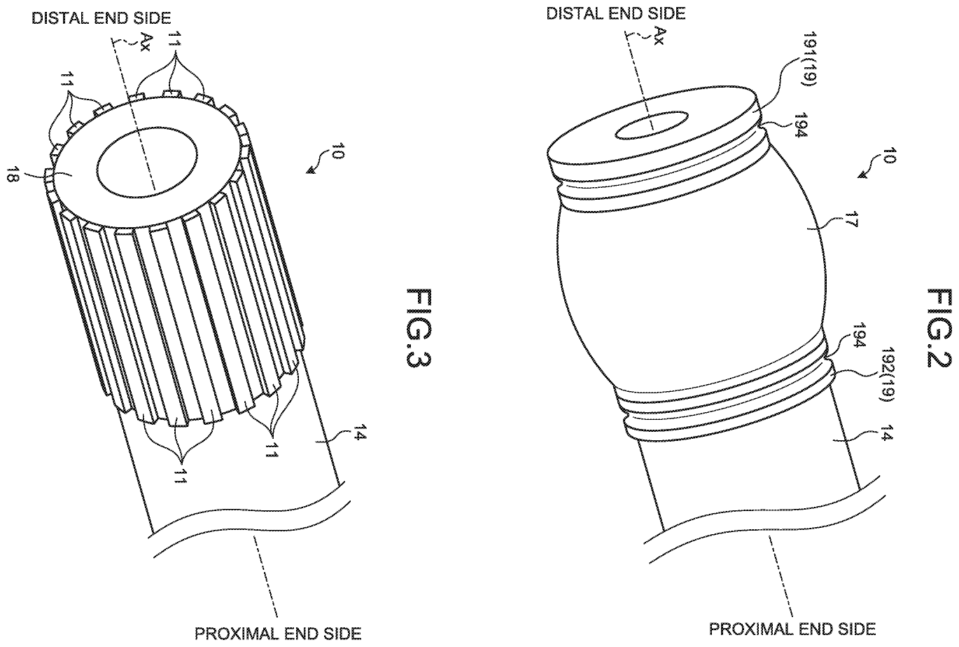

FIG. 2 to FIG. 5 are diagrams illustrating the configuration of the ultrasonic transducer 10.

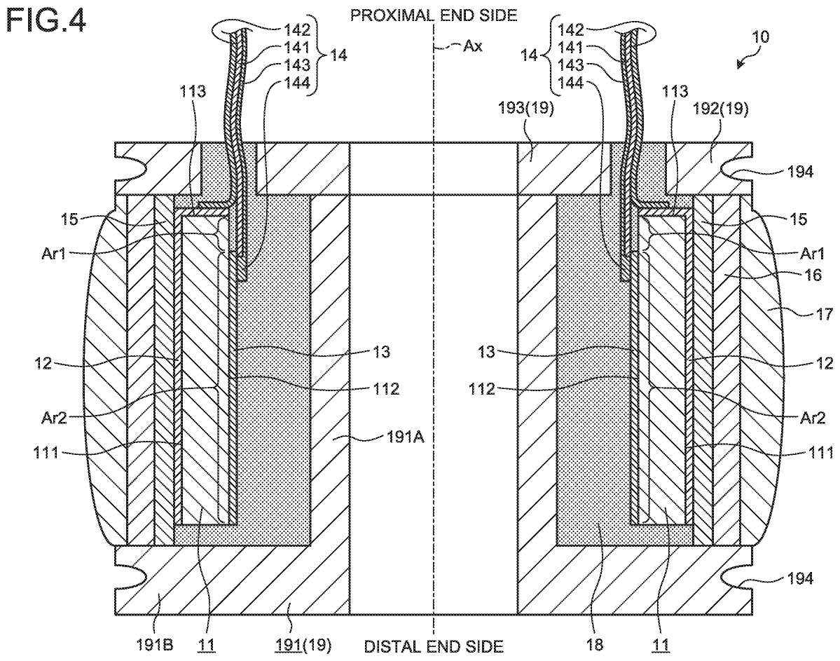

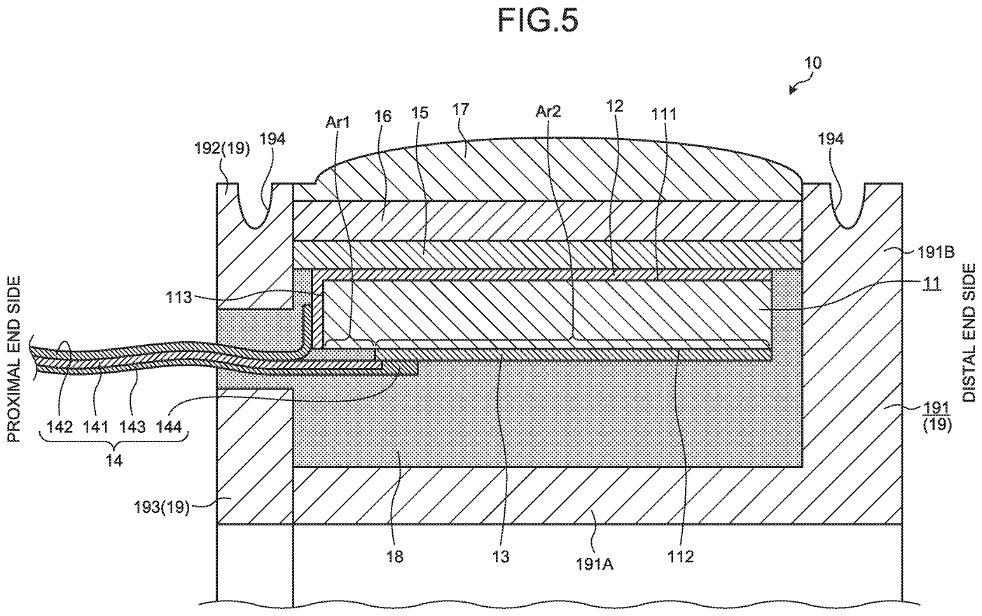

Specifically, FIG. 2 is a perspective view of the ultrasonic transducer 10 viewed from the distal end side. FIG. 3 is a perspective view of the ultrasonic transducer 10, where first electrodes 12, second electrodes 13, first acoustic matching layers 15, a second acoustic matching layer 16, an acoustic lens 17, and a locking member 19 are omitted. FIG. 4 is a cross-sectional view of the ultrasonic transducer 10 taken along a plane along an insertion axis Ax. FIG. 5 is a partial enlarged view of FIG. 4.

The ultrasonic transducer 10 is an ultrasonic transducer (an array ultrasonic transducer) of an electronic radial scan system, and includes a plurality of piezoelectric elements 11 (FIG. 3) that are regularly arrayed so as to form a cylinder. The ultrasonic transducer 10 radially transmits ultrasonic pulses from the cylinder, and performs scanning with the ultrasonic pulses in a rotation direction of 360 degrees about a central axis of the cylinder.

Further, as illustrated in FIG. 2 to FIG. 5, the ultrasonic transducer 10 includes the plurality of piezoelectric elements 11 (FIG. 3 to FIG. 5), the plurality of first electrodes 12 and second electrodes 13 (FIG. 4 and FIG. 5), a flexible printed board 14 (hereinafter, referred to as the FPC board 14), the first acoustic matching layers 15 and the second acoustic matching layer 16 (FIG. 4 and FIG. 5), the acoustic lens 17 (FIG. 2, FIG. 4, and FIG. 5), a backing member 18 (FIG. 3 to FIG. 5), and the locking members 19 (FIG. 2, FIG. 4, and FIG. 5).

As illustrated in FIG. 3 to FIG. 5, the piezoelectric elements 11 are constructed with elongated rectangular solids that extend in a straight manner in a direction along the insertion axis Ax (the direction corresponds to a first direction according to the present disclosure). Furthermore, as illustrated in FIG. 4 or FIG. 5, the first electrodes 12 and the second electrodes 13 are disposed on outer surfaces of the piezoelectric elements 11. The piezoelectric elements 11 convert a pulse signal (corresponding to an electrical signal according to the present disclosure) input via the transducer cable (not illustrated), the FPC board 14, and the second electrodes 13 as described above into an ultrasonic pulse, and transmit the ultrasonic pulses to the subject. Further, the piezoelectric elements 11 convert an ultrasonic echo reflected by the subject into an electrical echo signal (corresponding to the electrical signal according to the present disclosure) that represents the ultrasonic echo by a voltage change, and outputs the echo signal.

Here, the piezoelectric elements 11 are constructed with a PMN-PT single crystal, a PMN-PZT single crystal, a PZN-PT single crystal, a PIN-PZN-PT single crystal, or a relaxer material.

Meanwhile, the PMN-PT single crystal is an abbreviation of a solid solution of lead magnesium niobate and lead titanate. The PMN-PZT single crystal is an abbreviation of a solid solution of lead magnesium niobate and lead zirconate titanate. The PZN-PT single crystal is an abbreviation of a solid solution of lead zinc niobate and lead titanate. The PIN-PZN-PT single crystal is an abbreviation of a solid solution of lead indium niobate, lead zinc niobate, and lead titanate. The relaxer material is a generic term of a three-component piezoelectric material that is obtained by adding lead complex perovskite that is a relaxer material to lead zirconate titanate (PZT) in order to increase a piezoelectric constant or permittivity. The lead complex perovskite is represented by Pb(B1, B2)O₃, where B1 is any of magnesium, zinc, indium, and scandium, and B2 is any of niobium, tantalum, and tungsten. These materials have excellent piezoelectric effects. Therefore, these materials make it possible to reduce an electrical impedance value even when the size of a device is reduced, and are preferable from the viewpoint of impedance matching between the first electrodes 12 and the second electrodes 13.

The first electrodes 12 are constructed with a metal material or a resin material that has conductive property, and disposed on the outer surfaces of the piezoelectric elements 11 as described below.

Specifically, as illustrated in FIG. 4 or FIG. 5, the first electrodes 12 are disposed, in a continuous manner, on entire first surfaces 111, which are parallel to the insertion axis Ax and located on an outer surface side of the ultrasonic transducer 10 (a side away from a central axis of the cylindrical ultrasonic transducer 10), and entire intersecting surfaces 113, which are perpendicular to (or intersecting with) the first surfaces 111 (perpendicular to the insertion axis Ax) and located on the proximal end side, on the outer surfaces of the piezoelectric elements 11.

Further, the first electrodes 12 are electrically connected to a first conductive layer 142 that is arranged on the FPC board 14, and function as ground electrodes.

Similarly to the first electrodes 12, the second electrodes 13 are constructed with a metal material or a resin material that has conductive property, and disposed on the outer surfaces of the piezoelectric elements 11 as described below.

Specifically, as illustrated in FIG. 4 or FIG. 5, the second electrodes 13 are disposed on second surfaces 112 that are located opposite to the first surfaces 111 on the outer surfaces of the piezoelectric elements 11. More specifically, the second electrodes 13 are disposed in regions Ar2 (FIG. 4 and FIG. 5) except for regions Ar1 (FIG. 4 and FIG. 5) on the intersecting surfaces 113 side on the entire second surfaces 112. In other words, the second electrodes 13 are disposed so as to be separated from the first electrode 12.

Further, the second electrodes 13 are electrically connected to corresponding second conductive layers 143 that are arranged on the FPC board 14, and function as signal electrodes for inputting and outputting signals to and from the piezoelectric elements 11.

The FPC board 14 is a component that electrically connects the transducer cable (not illustrated) as described above to the first electrodes 12 and the second electrodes 13. As illustrated in FIG. 4 or FIG. 5, the FPC board 14 includes a substrate 141, the first conductive layer 142, the plurality of second conductive layers 143, and a bonding electrode 144.

The substrate 141 is a flexible substrate constructed with an insulating material, such as polyimide.

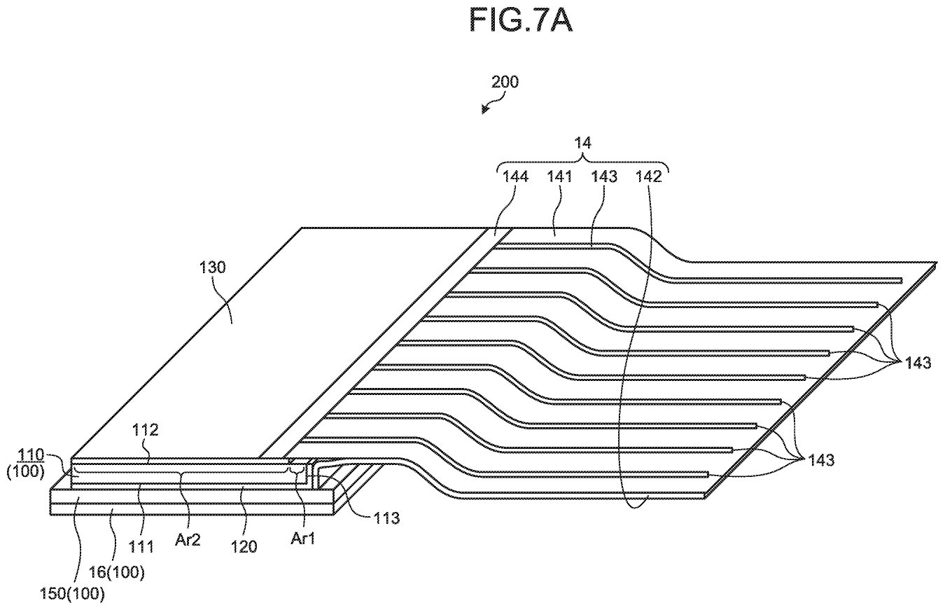

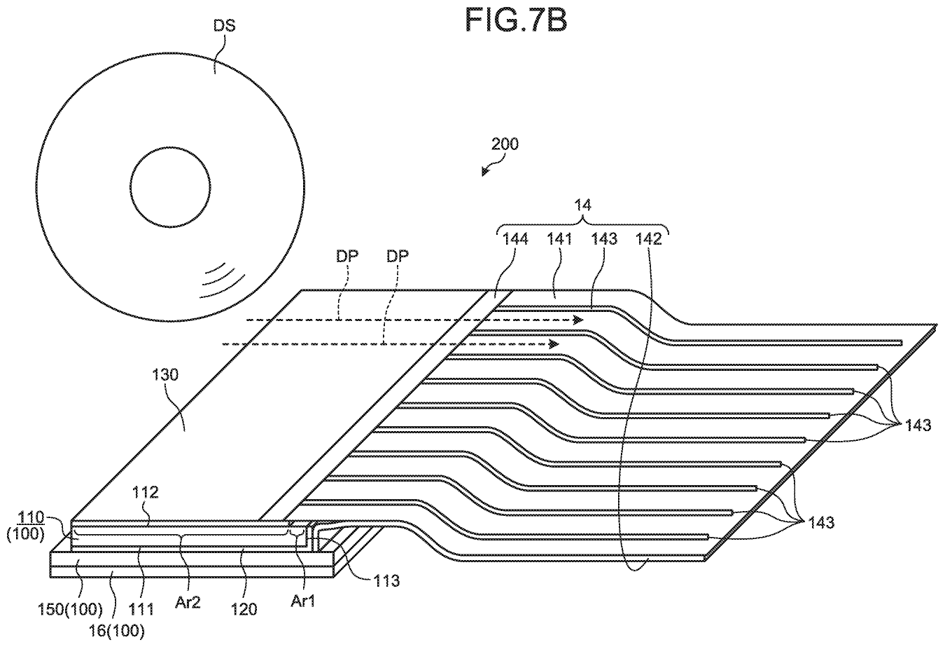

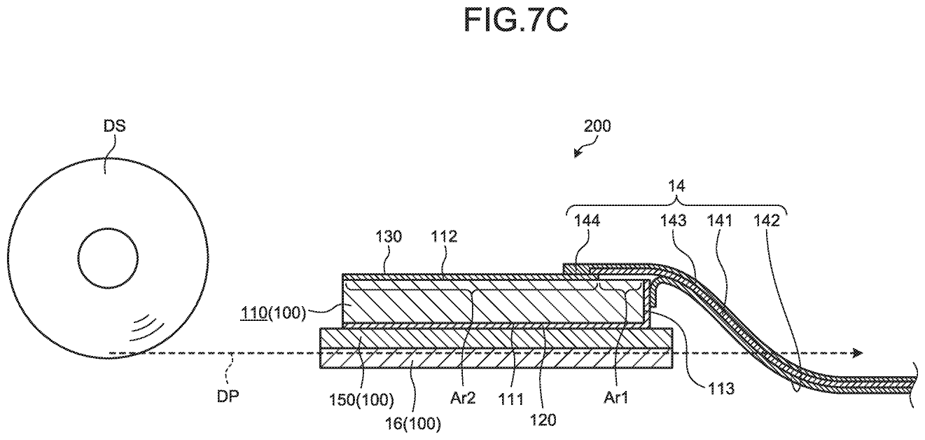

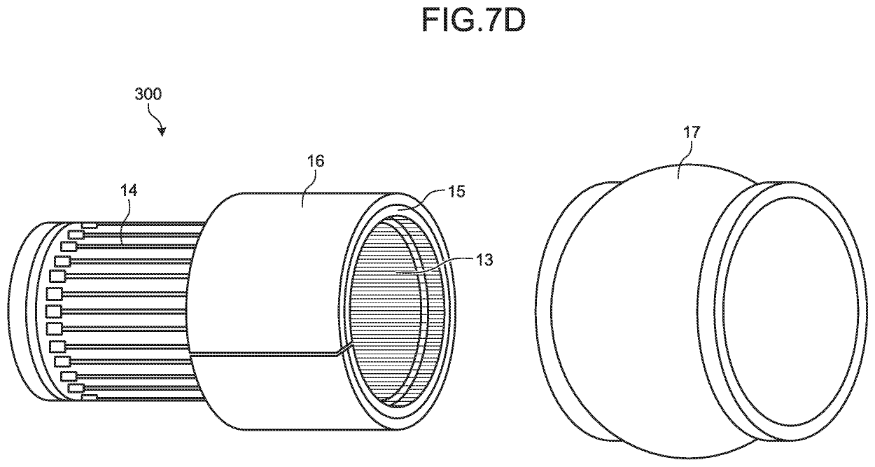

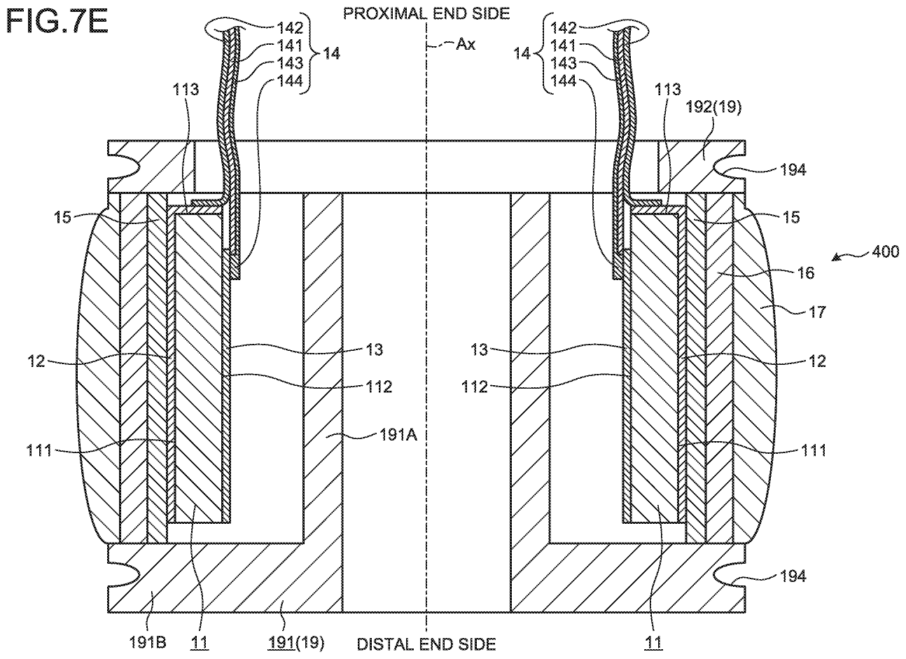

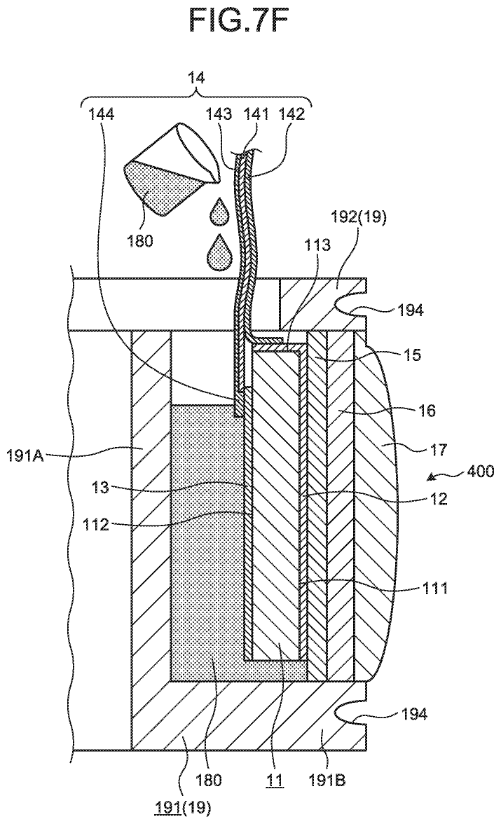

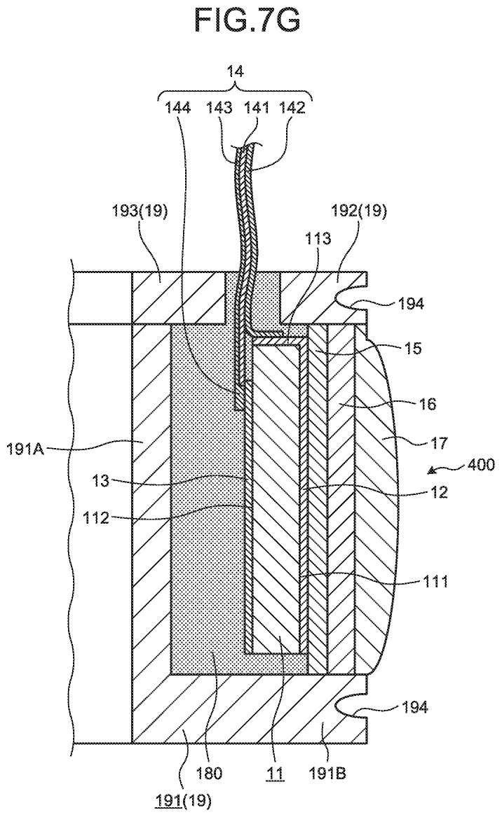

The first conductive layer 142 is a ground wiring serving as a ground, and uniformly arranged on one surface of the substrate 141 (see FIG. 7A). Further, the first conductive layer 142 is electrically connected to portions of the first electrodes 12 on the intersecting surfaces 113 in such a manner that a distal end side of the first conductive layer 142 is bent from the substrate 141. In other words, the first conductive layer 142 corresponds to a first wiring according to the present disclosure.

The plurality of second conductive layers 143 are signal wirings for transmitting the pulse signal and the echo signal as described above between the transducer cable (not illustrated) as described above and each of the second electrodes 13 disposed on the piezoelectric elements 11. Further, the plurality of second conductive layers 143 are configured as wiring patterns so as to be arranged in a direction perpendicular to a longitudinal direction of the FPC board 14 on the other surface that is located opposite to the above-described one surface of the substrate 141 (see FIG. 7A).

While details are not illustrated in the figures, protective layers that are constructed with an insulating material, such as polyimide, and protect the first conductive layer 142 and the second conductive layers 143 are disposed on the above-described one surface and the other surface of the substrate 141.

The bonding electrode 144 is disposed on a distal end of the FPC board 14, extends in a direction perpendicular to the longitudinal direction of the FPC board 14, and is electrically connected to each of the second conductive layers 143 (see FIG. 7A). Further, the bonding electrode 144 is electrically connected to portions of the second electrodes 13 extending to the intersecting surfaces 113 side relative to the centers of the second surfaces 112. In other words, the second conductive layers 143 are electrically connected to the second electrodes 13 via the bonding electrode 144, and correspond to second wirings according to the present disclosure.

The first conductive layer 142, the second conductive layers 143, and the bonding electrode 144 as described above are constructed with, for example, a conductive material, such as copper.

As illustrated in FIG. 4 or FIG. 5, the first acoustic matching layers 15 are disposed on the corresponding first surfaces 111 side of the piezoelectric elements 11. The second acoustic matching layer 16 is constructed with a different material from that of the first acoustic matching layers 15, and disposed on the outer surface side of the ultrasonic transducer 10 relative to the first acoustic matching layers 15.

More specifically, the first acoustic matching layers 15 and the second acoustic matching layer 16 are members that match acoustic impedance between the piezoelectric elements 11 and a subject in order to effectively transmit sounds (ultrasonic waves) between the piezoelectric elements 11 and the subject.

Meanwhile, in the present embodiment, an example is described in which the two acoustic matching layers (the first acoustic matching layers 15 and the second acoustic matching layer 16) are used; however, it may be possible to use a single acoustic matching layer or three or more acoustic matching layers depending on characteristics of the piezoelectric elements 11 and the subject. Further, the ultrasonic transducer need not always include the acoustic matching layer as long as impedance matching with the subject is ensured.

The acoustic lens 17 is constructed with, for example, silicone resin or the like, has an approximately cylindrical shape with an outer peripheral surface bent in a convex manner as illustrated in FIG. 2, FIG. 4, or FIG. 5, and serves as the outer surface of the ultrasonic transducer 10. Further, the acoustic lens 17 has a function to converge ultrasonic pulses that are transmitted from the piezoelectric elements 11 via the first acoustic matching layers 15 and the second acoustic matching layer 16. Meanwhile, the acoustic lens 17 is optionally disposed, and it may be possible to omit the acoustic lens 17.

As illustrated in FIG. 3 to FIG. 5, the backing member 18 is a member that is disposed on the second surfaces 112 side of the piezoelectric elements 11, and attenuates unnecessary ultrasonic vibration that occurs due to operation of the piezoelectric elements 11. The backing member 18 is constructed with a material having a large attenuation rate, such as epoxy resin in which alumina filler, zirconia filler, or the like is distributed, or rubber in which the above-described filler is distributed.

The locking member 19 is a portion that supports the members 11 to 18 as described above, and mounted on the rigid member 61. As illustrated in FIG. 2, FIG. 4, or FIG. 5, the locking member 19 includes a distal end locking member 191, a first proximal end locking member 192, and a second proximal end locking member 193.

As illustrated in FIG. 4, the distal end locking member 191 includes a cylindrical tubular body 191A that extends along the insertion axis Ax, and a flange portion 191B that is constructed by an annular plate body that is provided at a distal end of the tubular body 191A and extends outward (to the side away from a central axis of the tubular body 191A) at a right angle with respect to a distal end of the tubular body 191A.

As illustrated in FIG. 4, the first proximal end locking member 192 is constructed by an annular plate body that has a larger inner diameter dimension than an outer diameter dimension of the tubular body 191A and an approximately same outer diameter dimension as an outer diameter dimension of the flange portion 191B.

As illustrated in FIG. 4, the second proximal end locking member 193 is constructed by an annular plate body that has an approximately same inner diameter dimension as an inner diameter dimension of the tubular body 191A and a smaller outer diameter dimension than the inner diameter dimension of the first proximal end locking member 192. Meanwhile, a thickness dimension of the second proximal end locking member 193 is the same as a thickness dimension of the first proximal end locking member 192.

The second proximal end locking member 193 is disposed inside the first proximal end locking member 192. Further, the first proximal end locking member 192 and the second proximal end locking member 193 are disposed on a proximal end side of the tubular body 191A so as to face the flange portion 191B. Furthermore, the above-described members 11 to 18 are disposed in a space enclosed by the tubular body 191A, the flange portion 191B, the first proximal end locking member 192, and the second proximal end locking member 193.

Moreover, a balloon holding groove 194 for mounting a balloon (not illustrated) is formed on an outer peripheral surface of the flange portion 191B and an outer peripheral surface of the first proximal end locking member 192 so as to cover the acoustic lens 17.

Here, while details are not illustrated in the figures, an illumination lens that illuminates the inside of the subject with illumination light that is emitted from an emission end of the light guide along the insertion axis Ax, an objective optical system that condenses light (subject image) reflected inside the subject, and an imaging element that captures the subject image condensed by the objective optical system are disposed on the emission end side of the above-described light guide inside the cylindrical ultrasonic transducer 10 (inside the tubular body 191A). Further, an image signal captured by the imaging element is transmitted to the endoscope observation device 4 (the video processor 41) via the above-described signal cable.

In other words, the ultrasonic endoscope 2 according to the embodiment is constructed as a direct-viewing endoscope that observes a direction along the insertion axis Ax. Meanwhile, the ultrasonic endoscope 2 may be constructed by an oblique-viewing endoscope that observes a direction that intersects with the insertion axis Ax at an acute angle or a side-viewing endoscope that observes a direction perpendicular to the insertion axis Ax, instead of the direct-viewing endoscope.

Method of Manufacturing Ultrasonic Transducer

Next, a method of manufacturing the ultrasonic transducer 10 as described above will be described.

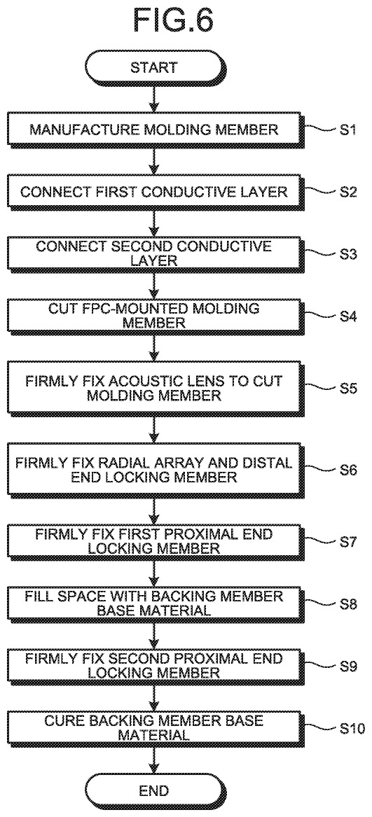

FIG. 6 is a flowchart illustrating a method of manufacturing the ultrasonic transducer 10. FIG. 7A to FIG. 7G are diagrams for explaining the method of manufacturing the ultrasonic transducer 10.

First, as illustrated in FIG. 7A, a molding member 100 is manufactured by laminating a first acoustic matching layer base material 150 and the second acoustic matching layer 16 in this order on one plate surface (corresponding to the first surface 111) side of a flat-plate piezoelectric element base material 110 (Step S1: molding member manufacturing process).

In other words, the first acoustic matching layer base material 150 and the second acoustic matching layer 16 correspond to a laminating member according to the present disclosure.

Here, the piezoelectric element base material 110 is made of a material from which the piezoelectric element 11 is produced, and has a shape of a flat plate in the form of a rectangle in a planer view as illustrated in FIG. 7A. Then, a first thin film 120, which is made of the same material as the first electrodes 12, is disposed entirely on the one plate surface (corresponding to the first surface 111) of the piezoelectric element base material 110 and entirely on another end surface (corresponding to the intersecting surface 113) perpendicular to (intersecting with) the one plate surface. Further, a second thin film 130, which is made of the same material as the second electrodes 13, is disposed in the region Ar2 except for the region Ar1 on the side near the above-described another end surface (corresponding to the intersecting surface 113) on the one plate surface (corresponding to the second surface 112) of the piezoelectric element base material 110.

Furthermore, the first acoustic matching layer base material 150 is a member that is made of the same material as the first acoustic matching layer 15.

Subsequently, as illustrated in FIG. 7A, the distal end side of the first conductive layer 142 is electrically connected, by soldering, to a portion of the first thin film 120 on the above-described another end surface (corresponding to the intersecting surface 113) in such a manner that the distal end side of the first conductive layer 142 is bent from the substrate 141 at an approximately right angle (Step S2: first wiring process).

Then, as illustrated in FIG. 7A, the bonding electrode 144 is electrically connected, by soldering, to a portion of the second thin film 130 extending to the above-described another end surface (corresponding to the intersecting surface 113) side relative to the center of the above-described other surface (corresponding to the second surface 112) (Step S3: second wiring process).

Meanwhile, the above described processes at Steps S1 to S3 need not always be performed in the above-described order, but may be started from any of the processes at Steps S1 to S3.

In the following, for convenience of explanation, the molding member 100 to which the FPC board 14 is connected through Steps S1 to S3 will be referred to as an FPC-mounted molding member 200 (FIG. 7A).

Subsequently, as illustrated in FIG. 7B or FIG. 7C, the FPC-mounted molding member 200 is cut by using a dicing saw DS (Step S4: molding process).

Specifically, a blade of a precise cutting machine, such as the dicing saw DS, is moved along respective cutting paths DP (FIG. 7B and FIG. 7C) that extend along the longitudinal direction (direction along the insertion axis Ax) of the FPC board 14 while rotating the blade, to thereby cut the molding member 100 to a middle depth of the second acoustic matching layer 16 and to a length that covers a part of the distal end side (including the bonding electrode 144) of the FPC board 14. As a result, each of the piezoelectric elements 11 (including the first electrodes 12 and the second electrodes 13) and each of the first acoustic matching layers 15 are formed. Further, by cutting the bonding electrode 144, the second conductive layers 143 are electrically disconnected from one another.

In the following, for convenience of explanation, the FPC-mounted molding member 200 that is cut at Step S4 will be referred to as a cut molding member 300 (FIG. 7D).

Subsequently, as illustrated in FIG. 7D, the cut molding member 300 is rounded into a cylindrical shape such that the plurality of piezoelectric elements 11 are arrayed so as to form a cylinder, and such that the second acoustic matching layer 16 is located on the outer peripheral side. Then, the cut molding member 300 that has been rounded is inserted into the acoustic lens 17, and the cut molding member 300 and the acoustic lens 17 are firmly fixed with each other (Step S5).

Meanwhile, while the acoustic lens 17 that is molded in advance is used at Step S5 in the present embodiment, embodiments are not limited to this example. For example, it may be possible to place the cut molding member 300 that has been rounded into a cylindrical shape into a mold, fills the mold with liquid resin material, and directly cast the acoustic lens 17 by cast molding on the cut molding member 300.

In the following, for convenience of explanation, a cylindrical unit in which the cut molding member 300 and the acoustic lens 17 are firmly fixed will be described as a radial array 400 (FIG. 7E).

Subsequently, as illustrated in FIG. 7E, the tubular body 191A is inserted into the cylindrical radial array 400 from the distal end side, and the radial array 400 and the distal end locking member 191 are firmly fixed while matching a center of the radial array 400 and a center of the distal end locking member 191 (Step S6).

Then, as illustrated in FIG. 7E, the FPC board 14 is inserted into the first proximal end locking member 192, and the radial array 400 and the first proximal end locking member 192 are firmly fixed while matching the center of the radial array 400 and a center of the first proximal end locking member 192 (Step S7).

Subsequently, as illustrated in FIG. 7F, a posture of a unit of the distal end locking member 191, the radial array 400, and the first proximal end locking member 192 that are firmly fixed with one another is changed, such that the first proximal end locking member 192 faces upward. Then, a space enclosed by the tubular body 191A, the flange portion 191B, and the radial array 400 is filled with a material (a backing member base material 180 (FIG. 7F)) that constructs the backing member 18 (Step S8).

Then, as illustrated in FIG. 7G, the second proximal end locking member 193 is firmly fixed to a proximal end portion of the tubular body 191A such that the FPC board 14 is inserted in a space between an inner peripheral surface of the first proximal end locking member 192 and an outer peripheral surface of the second proximal end locking member 193 (Step S9).

Finally, the backing member base material 180 filled at Step S8 is cured (Step S10), so that the ultrasonic transducer 10 is manufactured.

According to the ultrasonic transducer 10 of the embodiment as described above, the following effects are obtained.

In the ultrasonic transducer 10 according to the embodiment, the first electrode 12 is continuously disposed on two surfaces, such as the first surface 111 and the intersecting surface 113, in the piezoelectric element 11. Further, the second electrode 13 is disposed on the second surface 112 in the piezoelectric element 11. Furthermore, the first conductive layer 142 is electrically connected to a portion of the first electrode 12 on the intersecting surface 113. Moreover, the second conductive layer 143 is electrically connected to the second electrode 13.

Therefore, a joint portion between the first electrode 12 and the first conductive layer 142 and a joint portion between the second electrode 13 and the second conductive layer 143 are located away from the outer surface of the ultrasonic transducer 10. Consequently, it is possible to realize a structure in which the path length of the entry pathway of a medical agent or the like toward each of the joint portions via an interface (gap) between each of the distal end portions and the proximal end portions of the acoustic lens 17, the first acoustic matching layer 15, and the second acoustic matching layer 16 and the locking member 19 can be increased and the medial agent or the like is less likely to reach the above-described joint portions. In other words, it is possible to improve durability against a medical agent or the like.

Furthermore, it is not necessary to extend the first electrode 12 and the second electrode 13 to the proximal end side relative to the piezoelectric element 11. Therefore, it is possible to reduce the length of the ultrasonic transducer 10 in a direction along the insertion axis Ax. In other words, it is possible to improve insertion capability of the insertion portion 6 into the subject.

In view of the foregoing, according to the ultrasonic transducer 10 of the embodiment, it is possible to ensure the durability and reduce a size of the device.

Moreover, in the ultrasonic transducer 10 according to the embodiment, the first conductive layer 142 is a ground wiring that serves as a ground. Furthermore, the second conductive layer 143 is a signal wiring that supplies a pulse signal for causing the piezoelectric element 11 to emit an ultrasonic pulse. In other words, it is possible to arrange the joint portion of the signal wiring, for which it is necessary to take into account an influence on a medical agent or the like, at a position away from the outer surface of the ultrasonic transducer 10 relative to the joint portion of the ground wiring.

Moreover, in the ultrasonic transducer 10 according to the embodiment, the second conductive layer 143 is electrically connected to a portion of the second electrode 13 extending toward the intersecting surface 113 side relative to the center of the second surface 112. Therefore, it is possible to collectively arrange the first wiring and the second wiring according to the present disclosure from the intersecting surface 113 side to the proximal end side, so that it is possible to easily perform wiring. Furthermore, because it is possible to collectively arrange the first wiring and the second wiring, it is possible to construct the first wiring and the second wiring by the FPC board 14.

Moreover, in the method of manufacturing the ultrasonic transducer 10 according to the embodiment, the molding member manufacturing process is performed at Step S1, the first wiring process is performed at Step S2, the second wiring process is performed at Step S3, and thereafter the molding process is performed at Step S4.

Therefore, it is possible to easily perform wiring while realizing a narrow pitch, as compared to, for example, a case in which the plurality of piezoelectric elements 11 are molded by cutting the piezoelectric element base material 110 and thereafter sequentially connect a wiring to each of the first electrodes 12 and each of the second electrodes 13.

### OTHER EMBODIMENTS

While the embodiment of the present disclosure has been described above, the present disclosure is not limited to only the embodiment as described above.

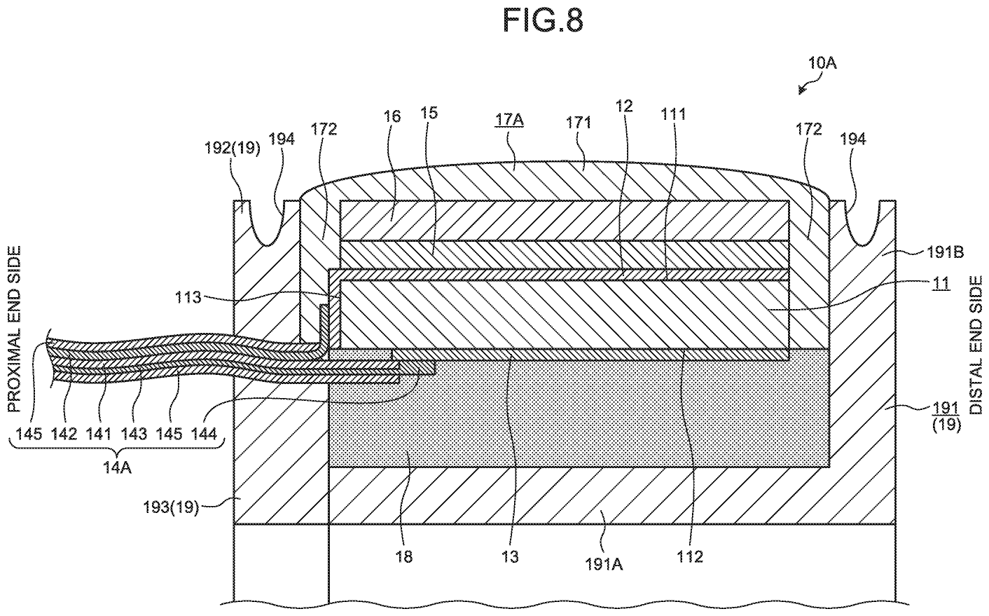

FIG. 8 is a diagram corresponding to FIG. 5, and illustrates an ultrasonic transducer 10A according to a first modification of the embodiment.

As illustrated in FIG. 8, in the ultrasonic transducer 10A according to the first modification, an acoustic lens 17A, instead of the acoustic lens 17, is adopted with respect to the ultrasonic transducer 10 (FIG. 5) according to the above-described embodiment.

The acoustic lens 17A is different from the acoustic lens 17 in terms of only a shape. As illustrated in FIG. 8, the acoustic lens 17A includes a lens unit 171 and a pair of protruding portions 172.

The lens unit 171 has an approximately cylindrical shape with an outer peripheral surface curved in a convex shape, and serves as an outer surface side of the ultrasonic transducer 10A relative to the second acoustic matching layer 16.

The pair of protruding portions 172 have annular shapes that are provided at and extended from both end portions of the lens unit 171 on the distal end side and the proximal end side at right angles toward a central axis of the lens unit 171. Further, as illustrated in FIG. 8, the pair of protruding portions 172 face both end portions of the plurality of piezoelectric elements 11, the plurality of first acoustic matching layers 15, and the second acoustic matching layer 16 on the distal end side and the proximal end side.

Furthermore, the first conductive layer 142 is electrically connected to a portion of the first electrode 12 on the intersecting surface 113 inside the acoustic lens 17A. Further, in an FPC board 14A according to the first modification, protective layers 145 are disposed on the one surface and the other surface of the substrate 141. The protective layers 145 are constructed with an insulating material, such as polyimide, and protect the first conductive layer 142 and the second conductive layer 143 such that only the joint portions of the first electrode 12 and the second electrode 13 are exposed.

According to the ultrasonic transducer 10A of the first modification, it is possible to further increase the path length of the entry pathway of a medical agent or the like toward the above-described joint portions via an interface (gap) between the acoustic lens 17A and the locking member 19. Therefore, it is possible to realize a structure in which a medical agent or the like is less likely to reach the above-described joint portions, so that it is possible to further improve durability against a medical agent or the like.

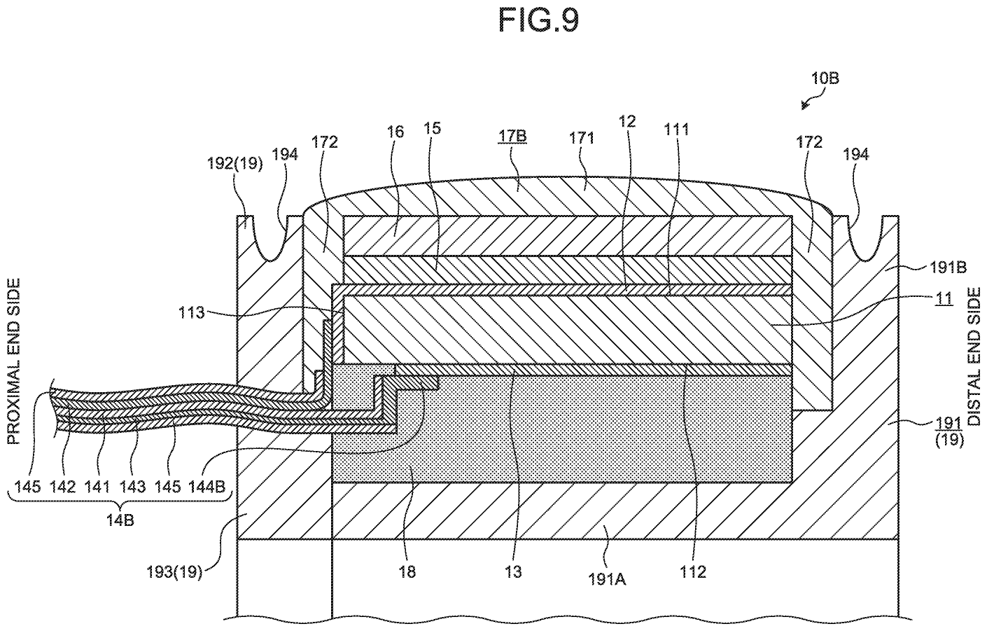

FIG. 9 is a diagram corresponding to FIG. 5, and illustrates an ultrasonic transducer 10B according to a second modification of the embodiment.

In the ultrasonic transducer 10B according to the second modification, as illustrated in FIG. 9, an acoustic lens 17B, instead of the acoustic lens 17, is adopted with respect to the ultrasonic transducer 10 (FIG. 5) according to the above-described embodiment.

As illustrated in FIG. 9, similarly to the acoustic lens 17A (FIG. 8) of the first modification as described above, the acoustic lens 17B includes the lens unit 171 and the pair of protruding portions 172.

The pair of protruding portions 172 according to the second modification have a longer protruding dimension than the pair of protruding portions 172 according to the first modification as described above. Further, as illustrated in FIG. 9, the pair of protruding portions 172 face both end portions of the backing member 18, in addition to both end portions of the plurality of piezoelectric elements 11, the plurality of first acoustic matching layers 15, and the second acoustic matching layer 16, on the distal end side and the proximal end side.

Furthermore, an FPC board 14B according to the second modification includes the protective layers 145 similarly to the FPC board 14A according to the first modification as described above, and includes a bonding electrode 144B that has a different shape from the bonding electrode 144, as compared to the FPC board 14 of the embodiment as described above. As illustrated in FIG. 9, the bonding electrode 144B is constructed so as to extend along the insertion axis Ax and have a cross-section in the form of an approximate L-shape that is bent and extended from a proximal end portion toward a central axis of the cylindrical ultrasonic transducer 10B.

According to the ultrasonic transducer 10B of the second modification, it is possible to further increase the path length of the entry pathway of a medical agent or the like toward the above-described joint portions via an interface (gap) between the acoustic lens 17B and the locking member 19, as compared to the ultrasonic transducer 10A according to the first modification as described above. Therefore, it is possible to realize a structure in which a medical agent or the like is less likely to reach the above-described joint portions, so that it is possible to further improve durability against a medical agent or the like.

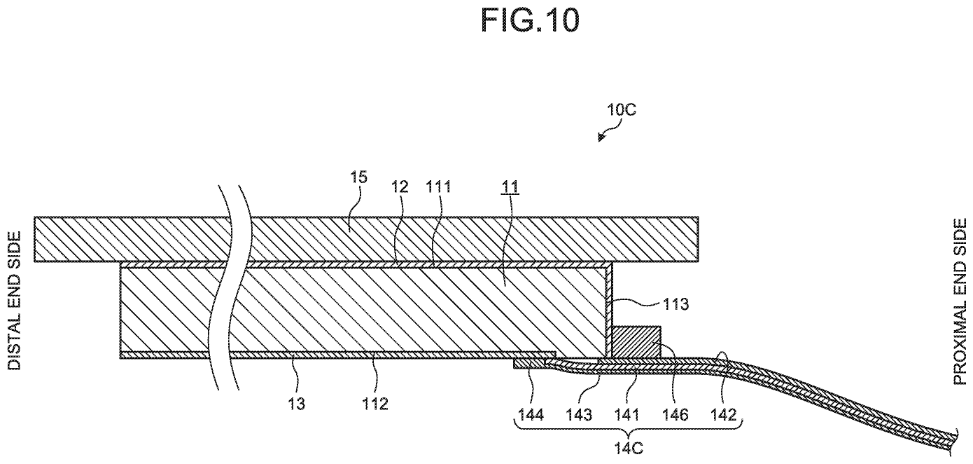

FIG. 10 is a diagram corresponding to FIG. 5, and illustrates an ultrasonic transducer 10C according to a third modification of the embodiment. Meanwhile, in FIG. 10, for convenience of explanation, the second acoustic matching layer 16, the acoustic lens 17, and the locking member 19 are omitted.

In the ultrasonic transducer 10C according to the third modification, as illustrated in FIG. 10, an FPC board 14C is adopted that is constructed by adding a bulged portion 146 to the FPC board 14 of the ultrasonic transducer 10 (FIG. 5) of the embodiment described above.

The bulged portion 146 is constructed with a conductive material, such as copper or nickel, and electrically connected to the first conductive layer 142. More specifically, the bulged portion 146 is bulged from the first conductive layer 142 so as to be away from the substrate 141, on the distal end side of the first conductive layer 142. Further, the bulged portion 146 is electrically connected to a portion of the first electrode 12 on the intersecting surface 113 while coming in contact with the portion. The bulged portion 146 is constructed by electroplating after completion of patterning of a signal wiring portion in the process of manufacturing the FPC board 14C. By using general photolithography, it is possible to construct the bulged portion 146 with precision down to a few micrometers (μm), so that it is possible to realize a conductive connection with accuracy.

According to the ultrasonic transducer 10C of the third modification, by bringing the bulged portion 146 in contact with the intersecting surface 113 (the first electrode 12) when the first wiring process at Step S2 and the second wiring process at S3 are performed, it is possible to easily determine the position of the FPC board 14C relative to the molding member 100. Further, it is possible to electrically connect, by soldering, the bonding electrode 144 and the bulged portion 146 while the position is determined, so that it is possible to easily perform wiring.

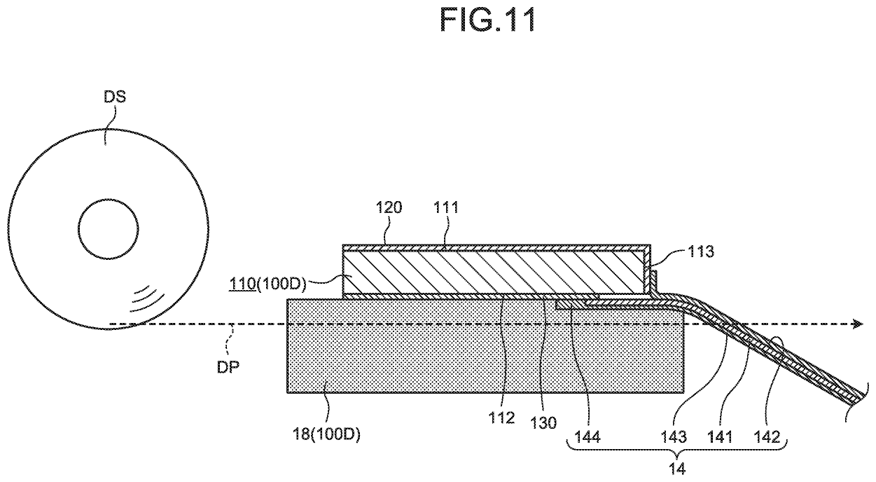

FIG. 11 is a diagram corresponding to FIG. 7C, and illustrates a molding member 100D according to a fourth modification of the embodiment.

In the embodiment as described above, in the molding member manufacturing process at Step S1, the molding member 100 is manufactured by sequentially laminating the first acoustic matching layer base material 150 and the second acoustic matching layer 16 on the first surface 111 side of the piezoelectric element base material 110, but embodiments are not limited to this example. It may be possible to adopt the molding member 100D (FIG. 11) of the fourth modification as the molding member of the present disclosure.

As illustrated in FIG. 11, the molding member 100D includes the backing member 18 that is disposed on the second surface 112 side of the flat-plate piezoelectric element base material 110.

In other words, the backing member 18 corresponds to the laminating member according to the present disclosure.

When the molding member 100D as described above is used, the first wiring process, the second wiring process, the molding member manufacturing process, and the molding process according to the present disclosure are performed as described below.

First, the distal end side of the first conductive layer 142 is electrically connected, by soldering, to a portion of the first thin film 120 on the intersecting surface 113 in such a manner that the distal end side of the first conductive layer 142 is bent from the substrate 141 at an approximately right angle (first wiring process).

Subsequently, the bonding electrode 144 is electrically connected, by soldering, to a portion of the second thin film 130 extending to the intersecting surface 113 side relative to the center of the second surface 112 (second wiring process).

Then, the molding member 100D is manufactured by disposing the backing member 18 on the second surface 112 side of the piezoelectric element base material 110 to which the FPC board 14 is connected (molding member manufacturing process).

Subsequently, as illustrated in FIG. 11, a blade of a precise cutting machine, such as the dicing saw DS, is moved along the cutting path DP that extends along the longitudinal direction (insertion axis Ax direction) of the FPC board 14 while rotating the blade, to thereby cut the molding member 100D to a middle depth of the backing member 18 and to a length that covers a part of the distal end side (including the bonding electrode 144) of the FPC board 14 (molding process). As a result, each of the piezoelectric elements 11 (including the first electrodes 12 and the second electrodes 13) is formed. Further, by cutting the bonding electrode 144, the second conductive layers 143 are electrically disconnected from one another.

In the embodiment and the first to fourth modifications of the embodiment as described above, the first wiring and the second wiring of the present disclosure are constructed by the flexible printed board; however, embodiments are not limited to this example, and at least one of the first wiring and the second wiring of the present disclosure may be construed by a lead wire or the like. Further, it may be possible to construct the first wiring and the second wiring of the present disclosure by different flexible boards.

In the embodiment and the first to fourth modifications of the embodiment as described above, the ultrasonic transducer of the present disclosure is applied to the ultrasonic endoscope 2; however, embodiments are not limited to this example. It may be possible to apply the ultrasonic transducer to an external ultrasonic probe that applies an ultrasonic pulse from a body surface of the subject.

In the embodiment and the first to fourth modifications of the embodiment as described above, an ultrasonic transducer of an electronic radial scan system is adopted as the ultrasonic transducer of the present disclosure; however, embodiments are not limited to this example. It may be possible to adopt an ultrasonic transducer of an electronic convex scan system or the like. Further, it may be possible to adopt a mechanical scan system, instead of an electronic scan system that electronically performs scan with the ultrasonic pulse.

In the embodiment and the first to fourth modifications of the embodiment as described above, the endoscope system 1 has both of the function to generate an ultrasonic image and the function to generate an endoscopic image; however, embodiments are not limited to this example. The endoscope system 1 may have only the function to generate an ultrasonic image.

In the embodiment and the first to fourth modifications of the embodiment as described above, the endoscope system 1 need not always be adopted in the medical field, but may be constructed as an endoscope system that observes inside of a subject, such as a mechanical structure, in an industrial field.

According to an ultrasonic transducer, an ultrasonic endoscope, and a method of manufacturing the ultrasonic transducer of the present disclosure, it is possible to ensure durability and reduce a size of the device.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic transducer that transmits and receives an ultrasonic wave, the ultrasonic transducer comprising:
a piezoelectric element that extends in a first direction and emits an ultrasonic wave in accordance with an input electrical signal, and converts an ultrasonic wave that is input from outside into an electrical signal;
a first electrode that is formed on a first surface of the piezoelectric element to be in parallel with the first direction, the first electrode including:
a first input portion through which the input electrical signal is to be input to the piezoelectric element, and
a first connection portion being formed continuously with the first input portion and intersecting the first direction,
wherein a first wiring is electrically connected to the first connection portion; and
a second electrode that is formed on a second surface of the piezoelectric element to be spaced apart from the first electrode, the second surface being oppose to the first surface in the piezoelectric element, the second electrode including:
a second input portion through which the input electrical signal is to be input to the piezoelectric element, and
a second connection portion being formed continuously with the second input portion,
wherein a second wiring is electrically connected to the second connection portion and arranged collectively with the first wiring.

2. The ultrasonic transducer according to claim 1, wherein
the piezoelectric element has a shape of an elongated rectangular solid that extends linearly in the first direction,
the first surface and the second surface are surfaces parallel to the first direction, and
the first connection portion of the first electrode is provided on a surface perpendicular to the first direction.

3. The ultrasonic transducer according to claim 1, wherein
the ultrasonic transducer is an array-type ultrasonic transducer in which a plurality of the piezoelectric elements are arrayed.

4. The ultrasonic transducer according to claim 3, wherein
the ultrasonic transducer is an ultrasonic transducer of an electronic radial scan system in which the plurality of piezoelectric elements are regularly arrayed so as to form a cylinder.

5. An ultrasonic transducer unit comprising:
the ultrasonic transducer according to claim 1; and
a flexible printed substrate including an electrically insulating base material and an electrically conductive layer that serves as at least one of the first wiring and the second wiring, wherein the electrically conductive layer is bent with respect to the base material and electrically connected to either one of the first electrode and the second electrode.

6. The ultrasonic transducer unit according to claim 5, wherein
the flexible printed substrate further includes another electrically conductive layer, wherein
one of the electrically conductive layer and the another electrically conductive layer is provided on one surface of the flexible printed substrate and serves as one of the first wiring and the second wiring, and
the other of the electrically conductive layer and the another electrically conductive layer is provided on the other surface of the flexible printed substrate, the other surface being oppose to the one surface, and serves as the other of the first wiring and the second wiring.

7. The ultrasonic transducer unit according to claim 6, wherein
an electrically conductive layer serving as the first wiring among the electrically conductive layer and the another electrically conductive layer is provided with a bulge portion that is electrically conductive to and bulged from the electrically conductive layer serving as the first wiring, wherein the bulge portion is in contact with and electrically connected to the first connection portion of the first electrode.

8. The ultrasonic transducer unit according to claim 5, wherein
the first wiring is a ground wiring serving as ground, and
the second wiring is a signal wiring through which an electrical signal that causes the piezoelectric element to emit the ultrasonic wave is input to the piezoelectric element.

9. The ultrasonic transducer unit according to claim 5, wherein
the second electrode is formed to extend toward the first connection portion beyond a center of the second surface, on the second surface, and
the second wiring is connected to a part of the second electrode, the part being beyond the center of the second surface toward the first connection portion.

10. An ultrasonic endoscope comprising:
an insertion portion to be inserted into a subject to be examined; and
the ultrasonic transducer unit according to claim 5, the ultrasonic transducer unit being provided at a distal end portion of the insertion portion.

11. The ultrasonic endoscope according to claim 10, wherein
the piezoelectric element is disposed such that the first connection portion faces toward a proximal end portion of the insertion portion.

12. A method of manufacturing an ultrasonic transducer comprising:
electrically connecting a first wiring on a portion of a first thin film that is continuously formed on a first surface and an intersecting surface of a piezoelectric element base material to constitute a piezoelectric element, the intersecting surface intersecting with the first surface, the portion corresponding to a first connection portion provided on the intersecting surface, the first thin film being to serve as a first electrode;
electrically connecting a second wiring on a second thin film formed on a second surface oppose to the first surface of the piezoelectric element base material, the second thin film being to serve as a second electrode;
manufacturing a molding member by forming a laminating member on one of the first thin film and the second thin film; and
producing a plurality of the piezoelectric elements by cutting the molding member to a middle depth of the laminating member after electrically connecting the first wiring, electrically connecting the second wiring, and manufacturing the molding member, wherein
at least one of the first wiring and the second wiring is an electrically conductive layer provided on a flexible printed substrate.
